(19) United States
(12) Patent Application Publication
(10) Pub. No.: US 2004/0253575 A1
(43) Pub. Date: Dec. 16, 2004

(54) Method for identifying a fuctional biological characteristic of a living matter

(75) Inventors: Michel Manfait, Verzy (FR); Dhruvananda Sockalingum, Reims (FR)

Correspondence Address: IP DEPARTMENT OF PIPER RUDNICK LLP, ONE LIBERTY PLACE, SUITE 4900, 1650 MARKET ST, PHILADELPHIA, PA, 19103, US

(73) Assignee: Bioalliance Pharma (S.A.), Paris (FR)

(21) Appl. No.: 10/331,678

(22) Filed: Dec. 27, 2002

Related U.S. Application Data
(63) Continuation of application No. PCT/FR01/02101, filed on Jun. 29, 2001.

(30) Foreign Application Priority Data
Jun. 29, 2000 (FR) 00/08440

(51) Int. Cl. C12Q 1/00

(52) U.S. Cl. 435/4

(57) ABSTRACT

A method for identification of a functional biological characteristic of a living material including a) subjecting at least one reference biological material for a functional biological to physical analysis Apr to establish a spectrum SAPr, b) calculating discriminant factors CPnr by a statistical analysis of all or part of the spectrum SAP, c) determining a specific functional descriptor Dfs of the functional biological characteristic from the discriminant factors Cpnr, d) subjecting the living material to be analyzed to steps (a) and (b), and e) comparing the discriminating factors CP of the living material to be analyzed with the specific functional descriptor Dfs to deduce a possible functional biological characteristic of the living material to be analyzed.

The invention pertains to a method for studying the multifactorial aspect of a biological function involving the use of physical technologies associated with biological methods taking more than one biological criterion into account. The invention makes it possible to identify the functional characteristics of living cells, tissues or microorganisms.

The method of the invention has numerous applications such as analysis of resistance phenomena (oncology and infectious diseases), identification of tissues and cells (histocytology, classification, primary or metastatic tumors), identification and analysis of microorganisms (Identification, Sensitivity/Resistance, Virulence, Epidemiology).

The evaluation of a particular function of a cell or tissue is presently based on a biological description. The description is established on morphological bases or on the detection of biochemical markers, verification of the expression of genes, verification of phenotypic expression, in vitro verification of the function or growth of a cell in the presence of drugs or specific markers. Extensive research and repeated examinations are required if all of these criteria are taken into account.

In the field of resistance to chemotherapy, numerous markers are described in the literature (Robert J., Multidrug resistance in oncology: diagnostic and therapeutic approaches, Europ J of Clin Investig (1999), 29, 536-545). Measurement is performed by biological techniques of immunohistochemical measurements of proteins linked to multiresistance phenomena (P-gp, MRP, LRP). Measurement of the functionality of these pumps (flux cytometry) is also proposed. Finally, measurement of the expression of the resistance gene (RT-PCR, flux cytometry) is performed (Marie J. P. et al., Multicentric evaluation of the MDR phenotype in leukemia, Leukemia 1997,11: 1095-1106).

The National Cancer Institute uses the function of expulsion of the proteins expressed in the case of resistance and thus measures the efflux of rhodamine by the P-gp pump on 60 cell culture lines. This is performed in a program entitled COMPARE (Alvarez M. et al., Generation of a drug resistance profile by quantification of mdrl/P-gp in the cell lines of the National Cancer Institute anticancer drug screen, J of Clinical Invest 1995, 95: 2205-2214). Nevertheless, this program is limited because the mechanism of induced resistance analyzed by this criterion is frequent but rarely the sole factor involved clinically.

Other resistance markers have been established by measuring the intracellular accumulation of endogenous substances or with therapeutic implications (glutathione, DNA adducts, drugs). These markers are dependent on the resistance mechanism and inductive drugs (Morjani H. et al., Anthracycline subcellular distribution in human leukemic cells by microspectrofluorometry: factors contributing to drug-induced cell death and reversal of multidrug resistance, Leukemia 1997,11: 1170-1179).

Biological methods for the evaluation of the susceptibility/resistance to chemotherapy have existed for many years. These methods are based on the concept of chemograms derived from the concept of antibiograms with a prediction of the sensitivity to drugs (Human Tumor Stem Cell Assay) in order to evaluate the growth in culture in the presence of varied chemical classes and to thereby define the susceptibility or resistance (Legrand O. et al., Simultaneous activity of MRP1 and P-gp is correlated with in vitro resistance to daunorubicin and with in vivo resistance in adult acute myeloid leukemia, Blood 1999, 94:1046-1056).

These methods are limited and are not widely used clinically because of difficulties in the sampling of the cells and their culturing. In fact, not all of the cells proliferate and it is difficult to obtain an agar culture that is representative of the cellular proliferation (35 to 60% can be evaluated) (Von Hoff D D., He's not going to talk about in vitro predictive assays again, is he?, J. Nat. Cancer Inst. 1990, 82: 96-101).

The result is that despite the increasingly perfected biological methods, none of them is accepted unanimously because they are limited by the criterion selected which is rarely representative of a cellular state or function. The implementation of data collection requiring multiple methods is limited by the successive multiplicity of the techniques that must be employed (genetic, immunologic chemical, analytical, culture).

Moreover, an evaluation by physical methods has already been described in the European patent application published as no. 0 568 126 using a confocal laser microscopy technique for determining the resistance or sensitivity to doxorubicin of cultured cells. In this patent application, only a different fluorescence image is observed in the membrane of resistant K562 cells. This image simply reflects the membranal alterations due to the accumulation of fluorescent doxorubicin in the P-gp pumps, overexpressed in the case of resistance. It represents a simple alternative to the immunohistochemical methods presented above.

The limits of this imaging method are linked, notably, to the disadvantages of studying the localization of the P-gp pump in the membrane on a single image. This membranal localization does not reflect in any way its function and cannot discriminate a state of resistance which, at the biological level, is multifactorial in humans.

Thus, the method described in this patent application does not make it possible to distinguish, e.g., a P-gp phenotype from a MRP1 phenotype.

Confocal fluorescence microscopy is also limited because the information obtained is often monoparametric or biparametric (measurement at one or two wavelengths in the fluorescence emission spectrum).

In the field of microbiology, there was described in U.S. Pat. No. 5,660,998 the use of a Fourier transformed infrared spectrometer to identify microorganisms. Identification is based on the global comparison of a single IR spectrum of the microorganism compared to a preestablished spectrum library. Although this single spectrum can lead to identification, it does not enable in its global nature definition of a biological function or characteristic associated with the microorganism (e.g., the sensitivity or resistance to a family of antibiotics).

The object of the invention is precisely to overcome the drawbacks of the methods of the prior art described above by offering the possibility of collecting simultaneously multiple pertinent criteria associated with the biological function under study. By means of mathematical modeling (multivariate statistical analysis, neuron networks, genetic algorithms, etc.), the method of the invention enables identification of the functional characteristics of living cells or tissues taking into account the multifactorial aspect of a function and thus simultaneously integrating multiple biological criteria.

This goal is attained according to the invention by means of a method for identification of a functional characteristic of a biological material comprising the following steps:

a) at least one reference biological material for a functional characteristic is subjected to physical analysis (Apr) to establish its spectrum (SAPr),

b) the discriminant factors (CPnr) are calculated by a statistical analysis of all or part of the spectrum SAP obtained in step (a),

c) a specific functional descriptor (Dfs) of the functional characteristic is established from said discriminant factors (CPnr),

d) the biological material to be analyzed is subjected to steps (a) and (b),

e) the discriminating factors (CP) of the biological material to be analyzed are compared with the specific functional descriptor (Dfs) obtained in step (c) in order to deduce a possible functional characteristic of the biological material to be analyzed.

Step (a) advantageously comprises the analysis of multiple reference biological materials presenting or not presenting the functional characteristic.

The method of the invention is remarkable in that it employs an integrated model based on the construction of the specific functional descriptor Dfs of the biological state. For example, with regard to resistance, multiple factors can be analyzed simultaneously. Thus, the integrated descriptor of the biological state will be constructed to be representative of the in vivo function and to augment the predictivity of the response. It will take into account in a single analysis the multifactorial aspect existing in human clinical practice.

This descriptor technique makes it possible notably to rapidly integrate a new criterion that is useful in clinical practice.

Thus, working with cells, microorganisms or living tissues, the method of the invention enables on the basis of multiple molecular criteria collected by means of physical analysis (Apr) determination of one or more functional biological characteristics and thus definition of a specific functional descriptor (Dfs) of it (them).

The biological material analyzed by the method of the invention can be a cell or tissue sample or even a single cell. The material, for example, can be comprised of tumor cells stemming either from cultures or from patients after collection of blood samples or by tissue biopsy and subsequently isolated by density gradient.

In the case of tissues, the physical analyses can be performed either on extemporaneous anatomical pieces either in vivo (directly on the accessible tissues or following a surgical operation or via the endoscopic route).

The biological material analyzed by the method of the invention can also be a microorganism (bacteria, yeasts, fungi, etc.) obtained, e.g., from an infectious focus or during culturing (after inoculation on agar) enabling analysis of microcolonies as well.

These samples are maintained under survival conditions during the physical analyses (AP).

Compared to the analysis techniques of the prior art, the method of the invention presents the advantage of not requiring any prior labeling of the samples for the physical analyses (AP).

The physical analysis of reference biological material(s) (APr) and of the biological material to be analyzed (AP) of step (a) is advantageously performed by spectroscopy and optical microspectroscopy, more specifically with Raman vibrational, infrared and fluorescence emission spectroscopies, or a combination of these techniques, thus providing spectra (SAPr or SAP) containing molecular information.

The Raman spectra are obtained with excitatory laser radiations in the wavelength domain extending from the ultraviolet to the near infrared, more specifically at 364, 514, 633, 785 and 830 nm. The spectral domain studied extends from 200 to 4000 cm⁻¹.

The fluorescence spectra are also obtained with excitatory laser radiations in the wavelength domain extending from the ultraviolet to the near infrared (in the case of a multiphonic radiation), more particularly at 364, 514, 633 and 785 nm. The spectral domain studied covers a region from 200 to 400 nm.

For the Raman and fluorescence microspectroscopies, the selection of microscope objective magnification (for example, 100×) allows definition of the spatial resolution (0.5 μm) at the level of the cell or tissue sample the dimensions of which range from 10 μm to several mm (for example: 15 to 30 μm for cells, 40 to 100 μm for bacterial microcolonies, 100 to 2000 μm for tissues).

For transmission or reflection infrared absorption spectroscopy the analyzed spectral domain extends from 400 to 7000 cm⁻¹ (more particularly, from 400 to 4000 cm⁻¹).

In infrared microspectroscopy, the spectra are obtained with a magnification objective ranging from 8× to 60× (usually 36×) on samples ranging from 10 μm to several mm (for example: 15 to 30 μm for cells, 40 to 100 μm for bacterial microcolonies, 100 to 2000 μm for tissues).

For the Raman diffusion, infrared absorption and fluorescence emission spectroscopies, the spectrum acquisition times are comprised between 0.1 and 1000 seconds, more particularly from 1 to 100 seconds for the measurements associated with the construction of the Dfs.

The spectra of the reference biological materials and the biological material to be analyzed of the reference cells or microorganisms presenting or not presenting the targeted functional characteristic are recorded under the same conditions by the same techniques. Compared to the data obtained with the conventional methodologies, for example of biochemistry, cellular and molecular biology, flux cytometry and immunocytochemistry, requiring on average from 10⁵ to 10⁷ cells and analysis times of several tens of minutes to several days, the spectroscopic data are obtained from a panel of 10 to 100 isolated cells (more particularly 30) or from 1000 for microorganisms with analysis times of several seconds to several minutes (generally from 1 to 100 seconds).

Remarkably, the method of the invention makes it possible to associate multiple criteria to perform an analysis of the functional characteristic of the biological material:

For example, for defining the sensitivity or resistance character, it is possible to identify a first spectroscopic criterion from cells known to be sensitive or resistant and to associate it with a second spectroscopic criterion specific of a resistance state in relation to a particular substance (e.g., doxorubicin). This enables construction of a specific functional descriptor (Dfs) of a phenotype of specific resistance to the anticancer agent (e.g., P-gp-DOX).

In the case of a sample, it is possible not only to perform cell or tissue identification (tissue origin of the cell: breast, blood, prostate, bladder) but also to identify a function or state associated with these cells (for example: metastasizing power or not).

With regard to microorganisms, their nature, their identification and all other particular characteristics can be recorded and linked with other criteria (resistance/sensitivity, virulence or lack thereof).

The spectra collected in step (a) are then the object of multivariate statistical analyses by Principal Component Analysis (PCA) or PLS (Partial Least Squares) or by other suitable mathematical methods, such as, e.g., a Euclidian representation, a KNN method, a SIMCA method or a combination of these approaches, for identifying the discriminant factors. The PLS method is a linear regression method applicable when the predictive variables are collinear (Haaland D. and Thomas E., Partial Least Squares methods for spectral analysis, Anal Chem (1988), 60, 1193). The KNN method is a multivariate statistical method based on Principal Component Analysis and which consists of classifying unknown samples in relation to their proximity in multidimensional space with known samples (Adam J., 1995, Chemometrics in Analytical Spectroscopy, Cambridge, The Royal Society of Chemists). The SIMCA method (Soft Independent Modeling by Class Analogy) is a multivariate statistical method based on Principal Component Analysis which requires the construction of Principal Component Analysis models each describing reference classes (Frank I. and Lanteri S., 1989, Chemometrics and Intelligent Laboratory systems, 5, 247). This representation will enable identification and attribution of the discriminating spectroscopic elements to the various biological criteria being studied. In practice, a set of frequency intervals is retained for its discrimination profile adapted to the functional character being studied. Thus, the set of the most discriminant spectral elements enables construction of the specific functional descriptor of the biological functional characteristic being studied taking into account multiple functional biological phenomena or criteria.

In step (d), the biological material to be analyzed will be subjected to exactly the same procedure in steps (a) to (b) as that of the reference biological material(s) and then will be compared in step (e) to the functional descriptor obtained in step (c). This comparison advantageously consists of measuring the distance between the CPn of the reference biological material(s) and the CP of the biological material to be analyzed.

The biological material to be analyzed is thus projected into the factorial plane retained for the presentation of the results and will thereby be classified according to the functional characteristic being studied.

For example, in the case of characterization of a resistance phenotype from spectroscopic data, a set of spectra (Raman, infrared, fluorescence) is recorded on isolated tumor cells (in culture or isolated from patients). Using appropriate statistical methods, these data enable extraction of a subset of spectroscopic elements (e.g., intensity, frequency, polarization, life span). The combination of these elements remarkably enables construction of Dfs leading to a discrimination of two or more cell populations (e.g., sensitive or resistant) or subpopulations possessing a particular biological function (e.g., a specific resistance mechanism such as P-gp, MRP1, non-MDR, etc.).

The statistical study of the spectral differences is thus performed between the system expressing or not expressing the different biological criteria useful for the determination of the function. For example, for the problem of the resistance phenotype in cancer pathology, it is possible to integrate:

the origin of the cancerous cell (breast, leukemia, bladder, prostate, etc.),

the sensitivity or resistance character (cultures known to be sensitive or resistant),

the type of substance having induced the resistance: e.g., class of anthracyclines, vinca alkaloids, taxans, platins, these classes being known to bring into play multiple types of pumps or phenomena intervening in the function of resistance.

But the invention also finds applications in the matter of identification of other biological functions or states on:

eukaryote cells, such as, e.g., the differentiation state, the phases of the cell cycle, the pathways of signaling, apoptosis and necrosis, the aptitude for proliferation, invasive power, tumoral state, etc.,

microorganisms, such as, e.g., the sensitivity to a family or families of antibiotics, virulence, adhesion and mechanisms of infection, etc.

tissues which might be healthy, pathological, tumoral, pretumoral, presenting an aptitude for regeneration, an oxygenation state, etc.

Thus, the following applications of the method of the invention can be more particularly envisaged:

sensitivity/resistance, especially in relation to different classes of pharmacological agents,

identification of tissues and cells (organ of origin, histology, primary or metastatic tumors),

guiding the surgical act in the case of resection of a tumor,

identification of microorganisms (identification of the genus, species and strain, resistance, virulence),

identification of new antibacterial targets,

cell therapy: characterization of cellular function of dendritic cells,

prediction of a therapeutic response, creation of a Def enabling definition of the good responders and non-responders to a chemotherapy (predictive pharmacology and early diagnosis),

monitoring the individual therapeutic response for a new patient or upon relapse,

gradation of pathology,

identification of prognostic factors orienting the therapeutic choices (new progressive factors can be integrated).

Other advantages and characteristics of the invention will become clear from the description below of the attached figures pertaining to the use of spectroscopies for defining a functional descriptor associated with a biological characteristic consisting of multiple resistance to anticancer agents in different cell lines.

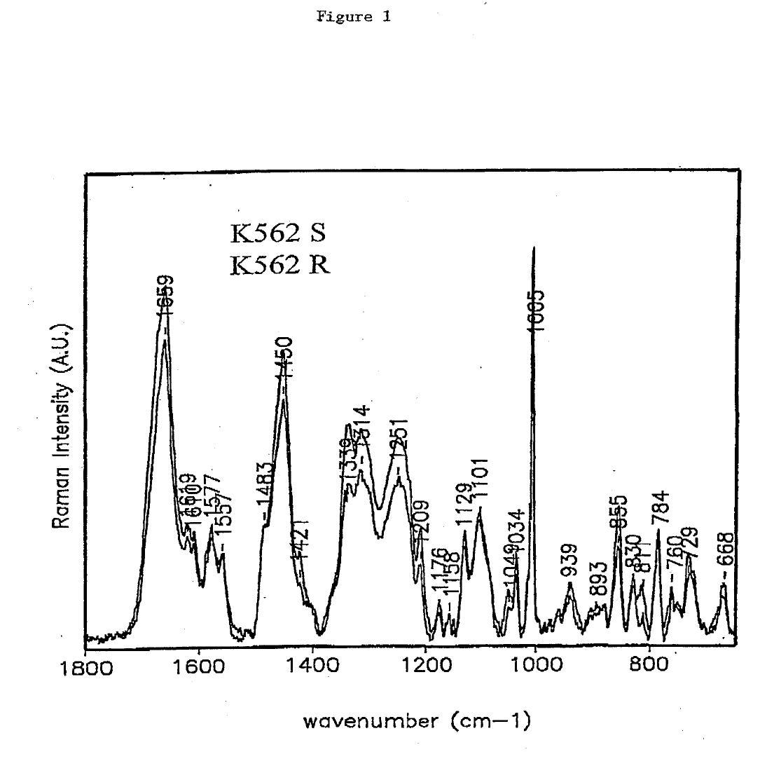

FIG. 1 represents the RAMAN spectra of sensitive (S) and resistant (R) K562 human leukemic cells with an MDR phenotype.

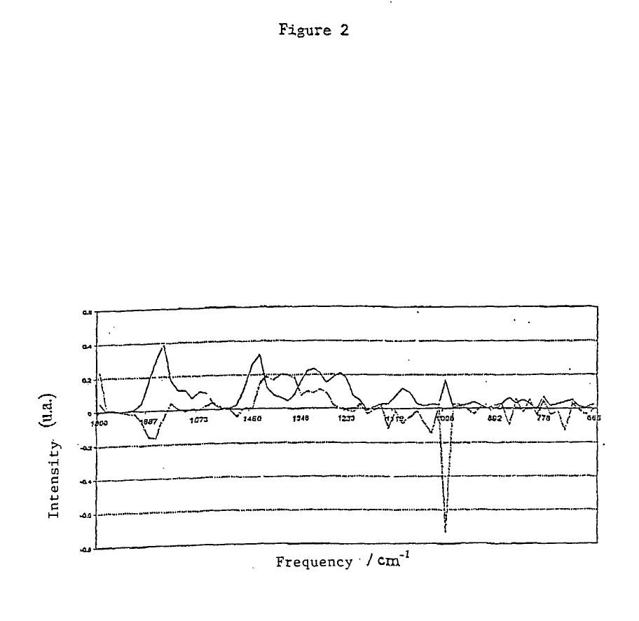

The spectra of FIG. 1 are subjected to a Principal Component Analysis (PCA). FIG. 2 shows an example of principal components which, after discriminant analysis for a biological function, will serve for the definition of Dfs and the 2D representation (factorial plane). FIG. 2 gives an example of 3 principal components for the construction of the specific functional descriptor of the resistance phenotype by discriminant analysis of the components CP1, CP2, CP3, . . . , CPn.

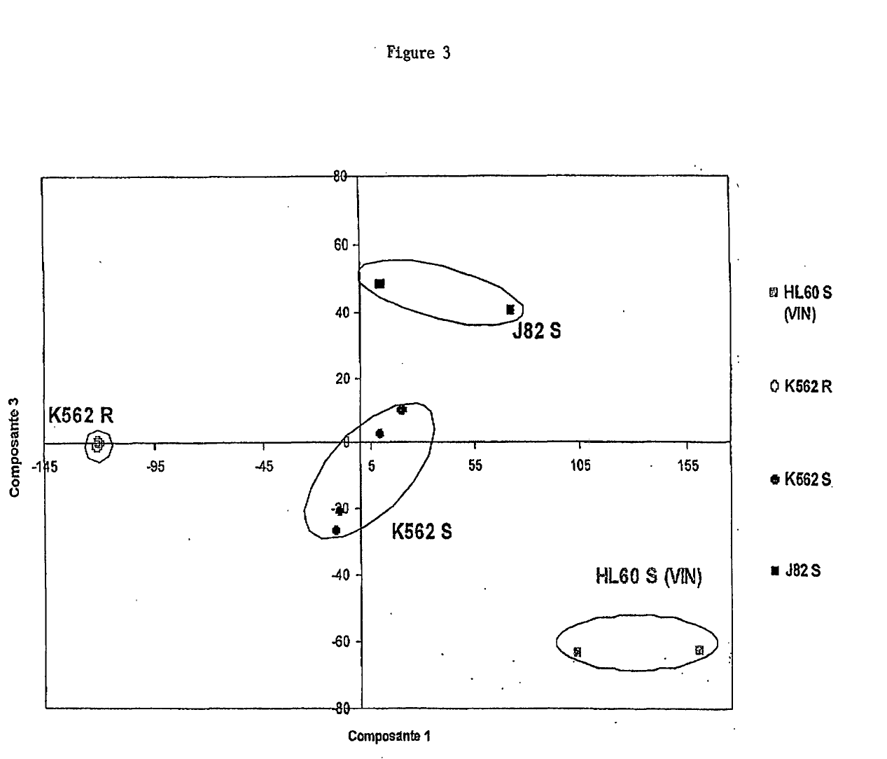

FIGS. 3, 4 and 5 represent a 2D or 3D projection (factorial plane) of the classification of the functional characteristic to be identified on the basis of its contribution in the initial spectral data.

FIG. 3 represents the identification in a 2D factorial plane (CP1 versus CP3) of sensitive K562, HL60 and J82 cells and resistant K562 cells (each system is individualized).

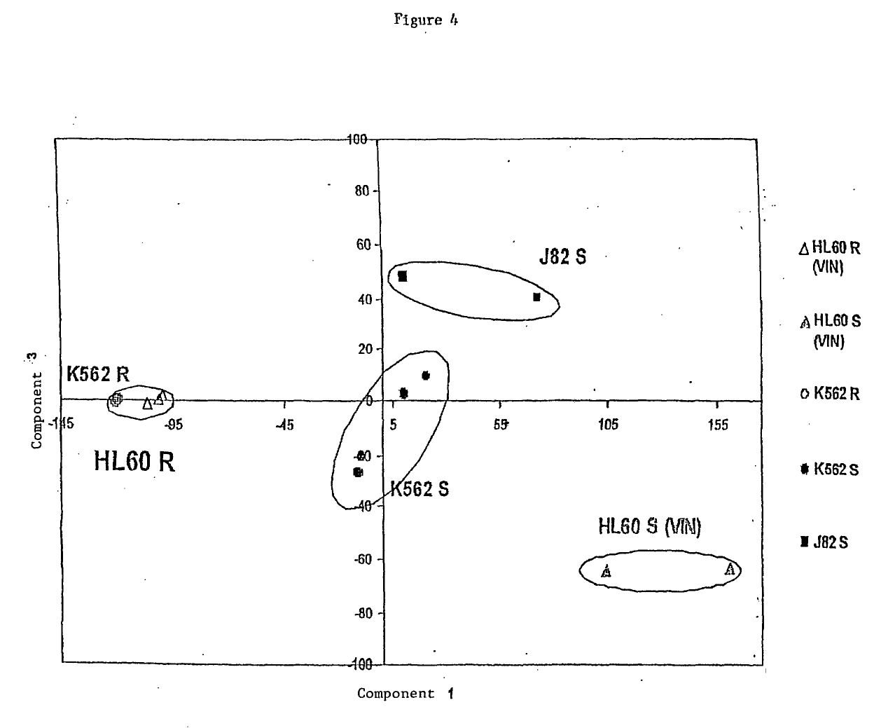

FIG. 4 represents the clustering of a new resistant HL60 line with the K562 R cluster. Although these lines are different, they cluster on the character “multiple resistance”. This shows the possibility of characterizing a precise biological function in different cell systems.

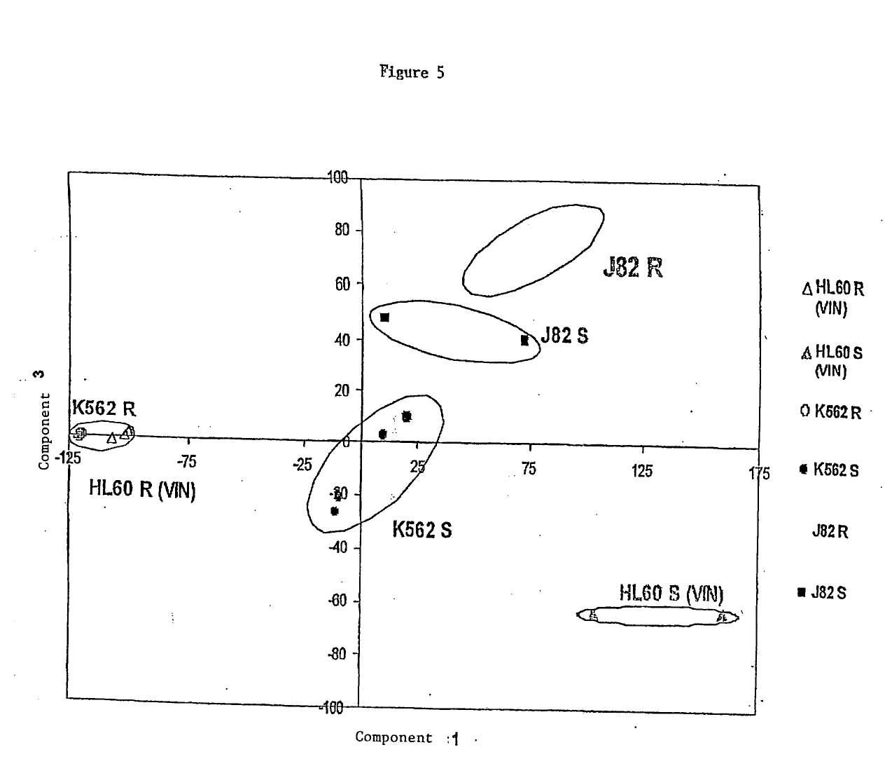

FIG. 5 represents a new resistant line J82 R which does not present the same resistance mechanism as the lines K562 R and HL60 R, and therefore is not brought into the same cluster.

1-8. (Canceled)

9. A method for identification of a functional biological characteristic of a living material comprising:
a) subjecting at least one reference biological material for a functional biological to physical analysis Apr to establish a spectrum SAPr,
b) calculating discriminant factors CPnr by a statistical analysis of all or part of the spectrum SAP,
c) determining a specific functional descriptor Dfs of the functional biological characteristic from the discriminant factors Cpnr,
d) subjecting the living material to be analyzed to steps (a) and (b), and
e) comparing the discriminating factors CP of the living material to be analyzed with the specific functional descriptor Dfs to deduce a possible functional biological characteristic of the living material to be analyzed.

10. The method according to claim 9, wherein the living material is a cell or tissue sample or a single cell.

11. The method according to claim 9, wherein physical measurements are performed by spectroscopy or microspectroscopy.

12. The method according to claim 9, wherein physical measurement are performed by Raman vibrational, infrared or fluorescence emission spectroscopies, or a combination thereof.

13. The method according to claim 12, wherein the Raman vibrational or fluorescence emission spectroscpies are performed with excitatory laser radiations in a wavelength domain extending from ultraviolet to near infrared.

14. The method according to claim 13, wherein the Raman vibrational or fluorescence emission spectroscpies are performed with excitatory laser radiations in a wavelength domain extending from ultraviolet to near infrared at 364, 514, 633 or 785 and in a spectral domain extending from 200 to 4000 cm⁻¹.

15. The method according to claim 12, wherein transmission or reflection infrared absorption spectroscopies are performed in a spectral domain extending from 400 to 7000 cm⁻¹.

16. The method according to claim 12, wherein transmission or reflection infrared absorption spectroscopies are performed in a spectral domain extending from 400 to 4000 cm⁻¹.

17. The method according to claim 11, wherein spectrum acquisition times of the Raman diffusion, infrared absorption and fluorescence emission spectroscopy measurements are comprised between 0.1 and 1000 seconds for measurements associated with construction of the integrated functional descriptor.

18. The method according to claim 11, wherein spectrum acquisition times of the Raman diffusion, infrared absorption and fluorescence emission spectroscopy measurements are comprised between 1 and 100 seconds for measurements associated with construction of the integrated functional descriptor.

19. The method according to claim 9, wherein the statistical analysis for characterizing the discriminant factors is a Principal Component Analysis (PCA) or PLS (Partial Least Squares) analysis or a Euclidean representation, a KNN method, a SIMCA method, or a combination thereof.
20. The method according to claim 9, wherein the functional biological characteristic is sensitivity or resistance to one or more pharmacological agents.
